(19) United States
(12) Patent Application Publication
(10) Pub. No.: US 2024/0300919 A1
(43) Pub. Date: Sep. 12, 2024

(54) A PROCESS FOR PREPARING BATEFENTEROL AND INTERMEDIATES THEREOF

(71) Applicant: GBR LABORATORIES PRIVATE LIMITED, HYDERABAD (IN)

(72) Inventors: Pratap Reddy Gaddam, Hyderabad (IN); Samhitha Reddy Gaddam, Hyderabad (IN); Madaalasa Reddy Gaddam, Hyderabad (IN); Udaya Kumar Reddy Mosali, Hyderabad (IN)

(73) Assignee: GBR LABORATORIES PRIVATE LIMITED, HYDERABAD (IN)

(21) Appl. No.: 18/277,340

(22) PCT Filed: Feb. 18, 2022
(86) PCT No.: PCT/IN2022/050143

§ 371 Date: Aug. 15, 2023

(30) Foreign Application Priority Data
Feb. 19, 2021 (IN) 202141004900

(51) Int. Cl. C07D 401/12

(52) U.S. Cl. CPC C07D 401/12

(57) ABSTRACT

The present invention relates to a process for preparation of Batefenterol of Formula (I) or pharmaceutically acceptable salts thereof, and the process for the preparation of the chiral intermediate compounds including compound of Formula (VII) by chiral sulfide mediated epoxidation. The process involves preparation of the intermediate compounds of Formula (VII), followed by preparation of Batefenterol from the intermediate compounds of Formula (VII) via intermediate compounds of Formula (X) and Formula (XI); or via intermediate compounds of Formula (IX) and Formula (XI); or via intermediate compounds of Formula (VII) and Formula (XI); or via intermediate compounds of the Formula (XV) and Formula (XVI); or via intermediate compounds of the Formula (XIX) and Formula (XVI). The process is cost effective and gives higher yield and better purity. The process of the present invention does not use hypertoxic materials and the final compound (I) prepared by the process is highly efficient and possess excellent powdery nature.

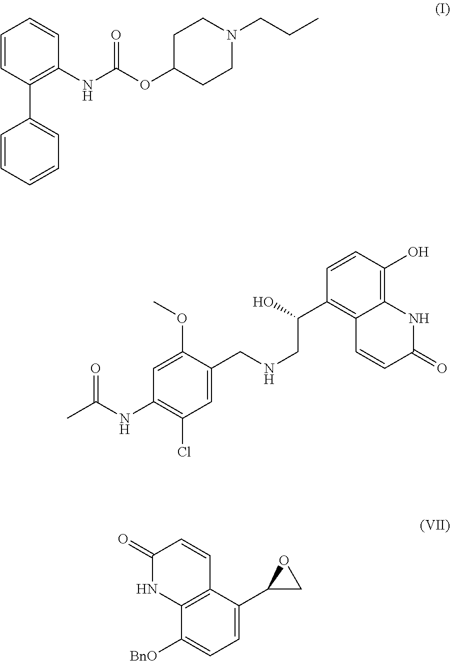

### CROSS REFERENCE TO RELATED APPLICATION

This application is a national phase entry of PCT Application No PCT/IN2022/050143, filed Feb. 18, 2022 titled “A PROCESS FOR PREPARING BATEFENTEROL AND INTERMEDIATES THEREOF” which claims priority to Indian Application No. 202141004900, filed Feb. 2, 2021 titled “A PROCESS FOR PREPARING BATEFENTEROL AND INTERMEDIATES THEREOF” which are incorporated herein by reference in their entirety.

### FIELD OF THE INVENTION

The present invention relates to a process for preparation of chiral intermediates of bi-functional muscarinic antagonist β2-agonist (MABAs) and more particularly to a process for preparation of chiral intermediates of Batefenterol by chiral sulfide mediated epoxidation.

### BACKGROUND OF THE INVENTION

Respiratory disorders are one of the leading causes of death in the world. Respiratory disorders are associated mainly with tobacco smoking, air pollution or occupational exposure, that can cause obstruction of airflow in the lungs resulting in bouts of breathlessness. COPD, bronchial asthma, chronic bronchitis, asthmatic bronchitis and emphysema are some of the respiratory disorders.

Bronchodilators are frequently used to treat respiratory disorders. The bronchodilators help loosen tight muscles of the airways leading to the widening of airways. The widening of airways in turn leads to easy breathing. Various class of compounds work as bronchodilators such as the β-adrenoceptor agonists, muscarinic receptor antagonists and the like. These bronchodilators are available in both short acting and long acting forms.

Of particular interest are the long-acting dual-pharmacology muscarinic antagonist/β2-adrenoceptor agonists (MABA). The MABAs function by combining muscarinic antagonism and β2-agonism in a single molecule. One such MABA is the Batefenterol. Batefenterol is currently under development as a long-acting bronchodilator. Due to the dual pharmacological activity, it is anticipated that Batefenterol would offer greater efficacy than single-mechanism long-acting muscarinic antagonists (LAMAs) or long-acting β2 receptor agonists (LABAs).

There is a growing interest in the development of cost effective and environmentally friendly processes for muscarinic antagonist/β2-adrenoceptor agonists (MABA). The compound Batefenterol can be derived from a class of compounds called Quinolinones. The patent U.S. Pat. No. 7,521,558B2 by Theravance Inc discloses a crystalline form of biphenyl compound, and a process of preparing the compound involving formation of the intermediates 2-Quinolinones. The patent application WO2006122788A1 by Almirall Prodesfarma SA et al describes 4-(2-amino-1-hydroxyethyl) phenol derivatives as ß2 adrenergic agonists involving formation of intermediates 2-Quinolinones. These are some of the known processes for the preparation of certain antagonist and their intermediates. However, the known processes involving the preparation of intermediates are expensive and have extended production time. There is need to for a process of preparation of chiral intermediates of Batefenterol that is cost effective, less time consuming and has fewer steps of synthesis. There is a further need of a process that avoids the usage of toxic reagents like borane derivative during the (chiral) selective reduction.

Further, there is a need for an industrially feasible process for preparation of Batefenterol and its chiral intermediates with improved yield and purity, thereby reducing production cost and time. Also, there is a need to synthesize Batefenterol from simple raw materials using safe and simple process.

### SUMMARY OF THE INVENTION

The present invention describes a process for preparation of Batefenterol compound having the Formula (I) or pharmaceutically acceptable salts thereof, and the process for the preparation of chiral intermediate compounds of Formula I.

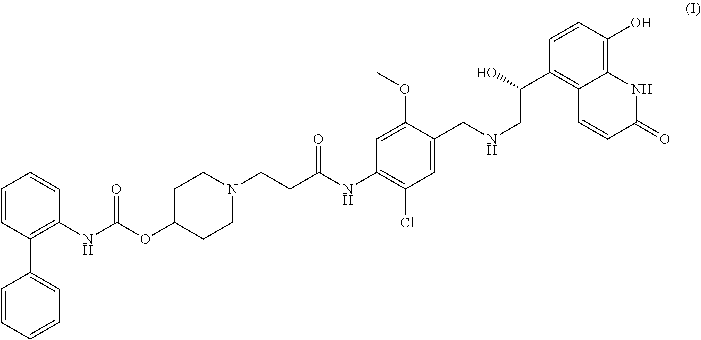 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

The process includes the steps of addition of the compound of Formula (II) to a solvent followed by adding a base and a benzylating agent to obtain a compound of Formula (III); followed by addition of the compound of Formula (III) to an acid and a brominating agent to make a reaction mixture; to obtain a compound of Formula (IV); carbonylation of compound of formula IV with strong base to give compound of formula V; chiral epoxidation of compound of formula V using chiral sulphide compound of Formula (VI) to form compound of formula VII; and synthesis of Batefenterol having the Formula (I) from the compound of Formula (VII) via intermediate compounds of Formula (X) and Formula (XI); or via intermediate compounds of the Formula (IX) and Formula (XI); or via intermediate compounds of the Formula (VII) and Formula (XI); or via intermediate compounds of the Formula (XV) and Formula (XVI); or via intermediate compounds of the Formula (XIX) and Formula (XVI).

In this process, carbonylation is carried out in presence of the solvents selected from tetrahydrofuran, methyl tert-butyl ether, diisopropyl ether, or diethyl ether; followed by treating with a strong base selected from n-butyl lithium, s-butyl lithium, lithium diisopropylamide, potassium bis(trimethylsilyl)amide; further followed by addition of a mixture of solvents, N, N-dimethylformamide and tetrahydrofuran in the ratio of 1:1 at the temperature of about-78° C. to 0° C.; chiral epoxidation is carried out using chiral sulphide derivative (VI), in presence of base selected from potassium hydroxide, sodium hydroxide, lithium hydroxide, in solvents selected from tert-butyl alcohol, isopropyl alcohol, methanol.

The process of synthesis of Batefenterol from intermediate compound of Formula (VII) via intermediate compounds of Formula (X) and Formula (XI) includes cleavage of chiral epoxide compound of formula VII is brominating agent to form compound of formula VIII; followed by optionally protection of compound of formula VIII in presence of protecting agents to form compound of formula IX; debenzylation of compound of formula IX in presence of debenzylating agent to form compound of formula X; and condensation of compound of formula X with compound of formula XI in presence of base.

In this process, the cleavage of chiral epoxide is carried out in solvents selected from Tetrahydrofuran, or halo solvents, and brominating agents selected from bromine, 1,3-dibromohydantoin, tetra n-butyl ammonium tri bromide, NBS; the protection is carried out using protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; the debenzylation is carried out using debenzylating agents selected from Pd/C, Pd/BaSO₄, or Raney nickel; the condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

The process of synthesis of Batefenterol from intermediate compound of Formula (VII) via intermediate compounds of the Formula (IX) and Formula (XI) includes cleavage of chiral epoxide compound of formula VII is brominating agent to form compound of formula VIII; followed by optionally protection of compound of formula VIII in presence of protecting agents to form compound of formula IX; condensation of compound of formula IX with compound of formula XI in presence of base to form compound of formula XII; and debenzylation of compound of formula XII in presence of debenzylating agent.

In this process, the cleavage of chiral epoxide is carried out in solvents selected from Tetrahydrofuran, or halo solvents, and the brominating agent is selected from bromine, 1,3-dibromohydantoin, tetra n-butyl ammonium tri bromide, NBS; the protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; the condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; the debenzylation is carried out using debenzylating agents selected from Pd/C, Pd/BaSO₄, or Raney nickel.

The process of synthesis of Batefenterol from intermediate compound of Formula (VII) via intermediate compounds of the Formula (VII) and Formula (XI) includes condensation of compound of formula VII with compound of formula XI in presence of base to form compound of formula XII; and debenzylation of compound of formula XIII in presence of debenzylating agent.

In this process, condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; and debenzylation is carried out using debenzylating agents selected from Pd/C, Pd/BaSO₄, or Raney nickel.

The process of synthesis of Batefenterol from intermediate compound of Formula (VII) via intermediate compounds of the Formula (XV) and Formula (XVI) includes cleavage of chiral epoxide compound of formula VII to form compound of formula XIV; followed by optionally protection of compound of formula XIV in presence of protecting agents to form compound of formula XV; condensation of compound of formula XV with compound of formula XVI in presence of base to form compound of formula XVII; and debenzylation of compound of formula XVII in presence of debenzylating agent.

In this process, cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS; protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃, debenzylation is carried out using debenzylating agents selected from Pd/C, Pd/BaSO₄, or Raney nickel.

The process of synthesis of Batefenterol from intermediate compound of Formula (VII) via intermediate compounds of the Formula (XIX) and Formula (XVI) includes cleavage of chiral epoxide compound of formula VII to form compound of formula XVIII; followed by optionally protection of compound of formula XVIII in presence of protecting agents to form compound of formula XIX; condensation of compound of formula XIX with compound of formula XVI in presence of base to form compound of formula XII; and debenzylation of compound of formula XII in presence of debenzylating agent.

In this process, cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS; protection is carried out by protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; debenzylation is carried out using debenzylating agents selected from Pd/C, Pd/BaSO₄, or Raney nickel.

### DETAILED DESCRIPTION OF THE INVENTION

The foregoing objects of the present invention are accomplished and the problems and shortcomings associated with the prior art, techniques and approaches are overcome by the present invention as described below in the preferred embodiments.

All materials used herein were commercially purchased as described herein or prepared from commercially purchased materials as described herein.

Although specific terms are used in the following description for sake of clarity, these terms are intended to refer only to particular structure of the invention selected for illustration in the drawings and are not intended to define or limit the scope of the invention.

References in the specification to “preferred embodiment” means that a particular feature, structure, characteristic, or function described in detail thereby omitting known constructions and functions for clear description of the present invention.

In one aspect, the present invention relates to a process for preparation of Batefenterol having the Formula (I) or pharmaceutically acceptable salts thereof.

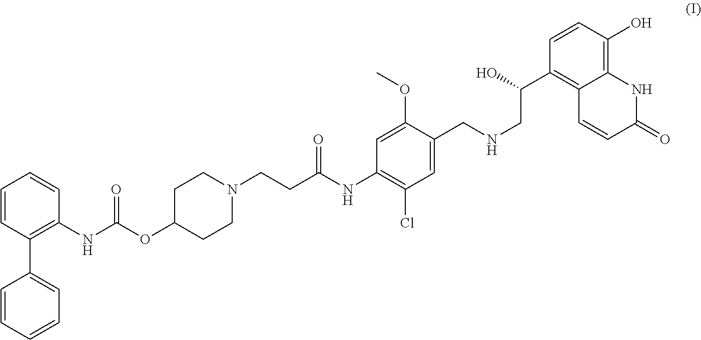 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

In another aspect, the present invention relates to a process for the preparation of chiral intermediate compounds including compound of the Formula (VII).

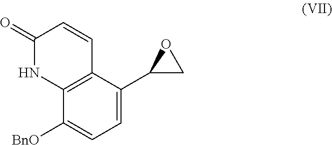 [SMILES: O=c1ccc2c([C@@H]3CO3)ccc(OCc3ccccc3)c2[nH]1]

In an embodiment, the preparation of the chiral intermediate compound of the Formula (VII) includes the steps of:
a) addition of the compound of Formula (II) to a solvent followed by adding a base and a benzylating agent to obtain a compound of Formula (III);

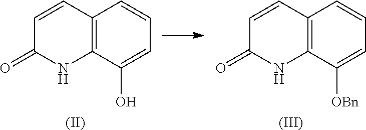 [SMILES: O=c1ccc2cccc(O)c2[nH]1 ; O=c1ccc2cccc(OCc3ccccc3)c2[nH]1 ; "(II)" ; "(III)"]

b) addition of the compound of Formula (III) to an acid and a brominating agent to make a reaction mixture; to obtain a compound of Formula (IV);

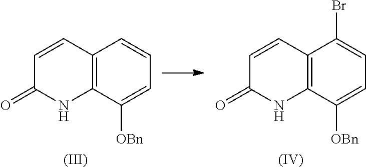 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]

c) carbonylation of compound of formula IV in presence of a strong base to give compound of formula V;

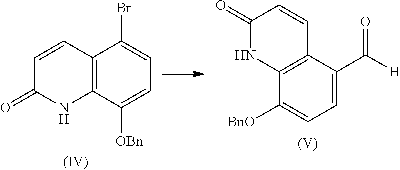

d) chiral epoxidation of compound of formula V, using chiral sulphide compound of formula (VI) to form compound of formula VII;

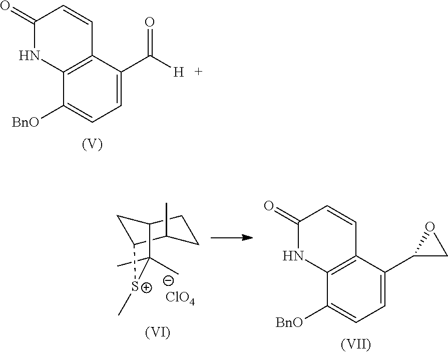

The detailed steps of the above-mentioned process are described herein:

In the step a), the compound of Formula (II) is 8-Hydroxyquinolin-2-(1H)-one. The solvent is selected from acetone, THF, DMF or like. The base is selected from potassium carbonate (K₂CO₃), Na₂CO₃, Cs₂CO₃ or like. The benzylating agent is selected from benzyl bromide, benzyl chloride, benzyl iodide, or like.

In the step b), the acid is selected from acetic acid, formic acid (HCO₂H), hydrobromic acid (HBr), or like. The brominating agent is selected from bromine, 1,3-dibromohydantoin, NBS or like. In the step c), the carbonylation is carried out in presence of the solvents selected from tetrahydrofuran, methyl tert-butyl ether, diisopropyl ether, or diethyl ether; followed by treating with a strong base selected from n-butyl lithium, s-butyl lithium, lithium diisopropylamide, potassium bis(trimethylsilyl)amide; further followed by addition of a mixture of solvents, N, N-dimethylformamide and tetrahydrofuran in the ratio of 1:1 at the temperature of about −78° C. to 0° C.

In the step d), the chiral epoxidation is carried out using chiral sulphide derivative (VI), in presence of base selected from potassium hydroxide, sodium hydroxide, lithium hydroxide, in solvents selected from tert-butyl alcohol, isopropyl alcohol, methanol.

The compound Batefenterol having the formula I is synthesized by various routes as described below:

The Route 1 of synthesis of compound of the Formula (I) from the intermediate compound of Formula (VII) via intermediates of the compound of Formula X and compound of XI includes the steps of:
a) cleavage of chiral epoxide compound of formula VII is brominating agent to form compound of formula VIII;

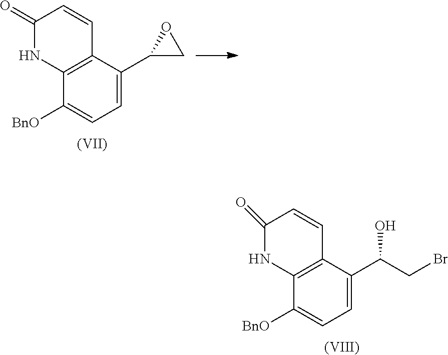

b) optionally protection of compound of formula VIII in presence of protecting agents to form compound of formula IX;

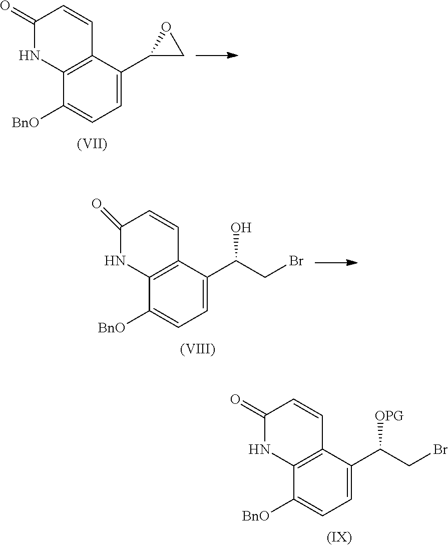

c) debenzylation of compound of formula IX in presence of debenzylating agent to form compound of formula X;

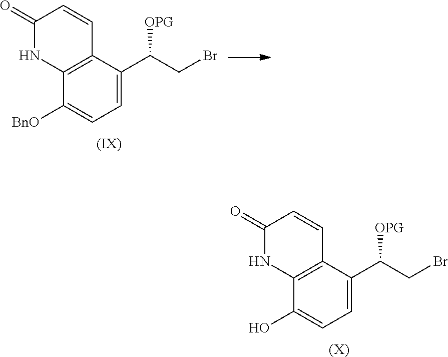

d) condensation of compound of formula X with compound of formula XI in presence of base.

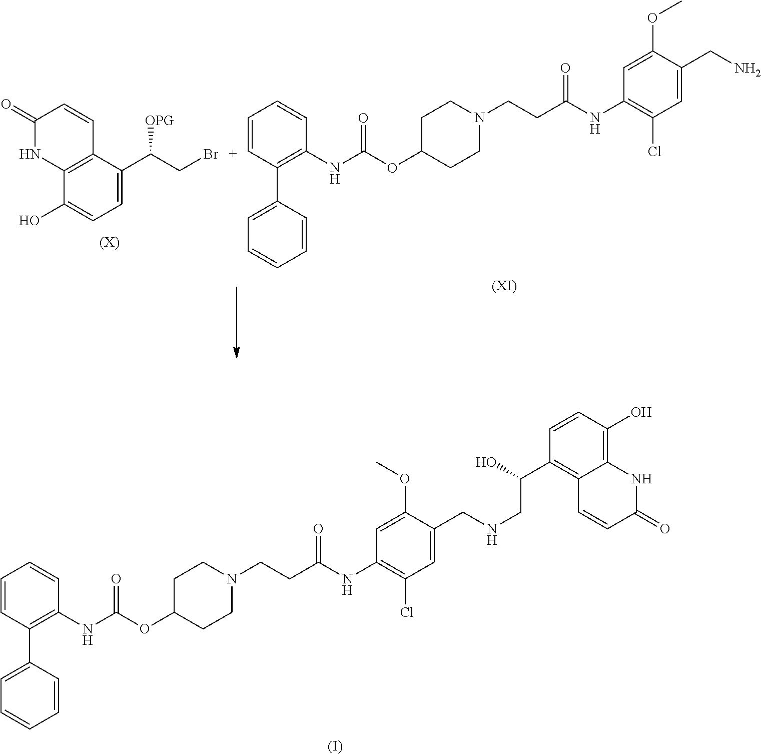

The detailed steps of the above-mentioned process as carried out under the solvent and reagent conditions are described herein:

In step a), the cleavage of chiral epoxide is carried out in solvents selected from Tetrahydrofuran, or halo solvents, and brominating agents selected from bromine, 1,3-dibromohydantoin, tetra n-butyl ammonium tri bromide, NBS.

In step b), the protection is carried out using protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole.

In step c), the debenzylation is carried out using debenzylating agents selected from Pd/C, Pd/BaSO₄, or Raney nickel.

In step d), the condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

The Route 2 of synthesis of compound of the Formula (I) from the intermediate of Formula (VII) via intermediates of the compound of Formula IX and compound of XI includes the steps of:
a) cleavage of chiral epoxide compound of formula VII is brominating agent to form compound of formula VIII;

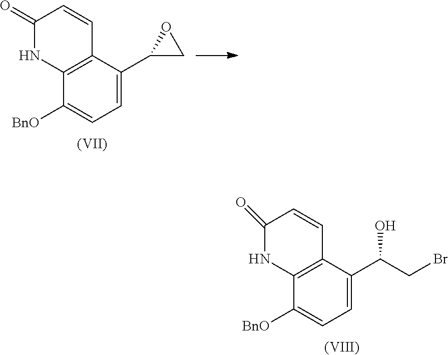

b) optionally protection of compound of formula VIII in presence of protecting agents to form compound of formula IX;

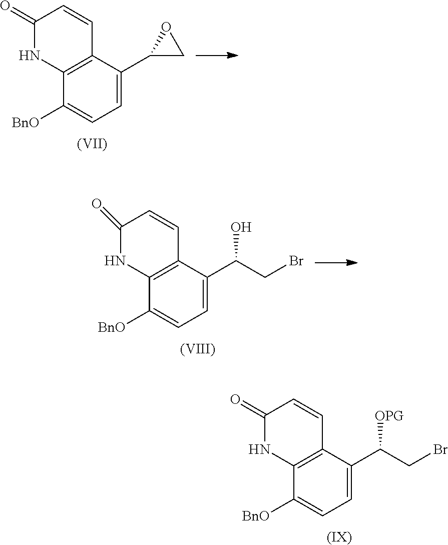

c) condensation of compound of formula IX with compound of formula XI in presence of base to form compound of formula XII;

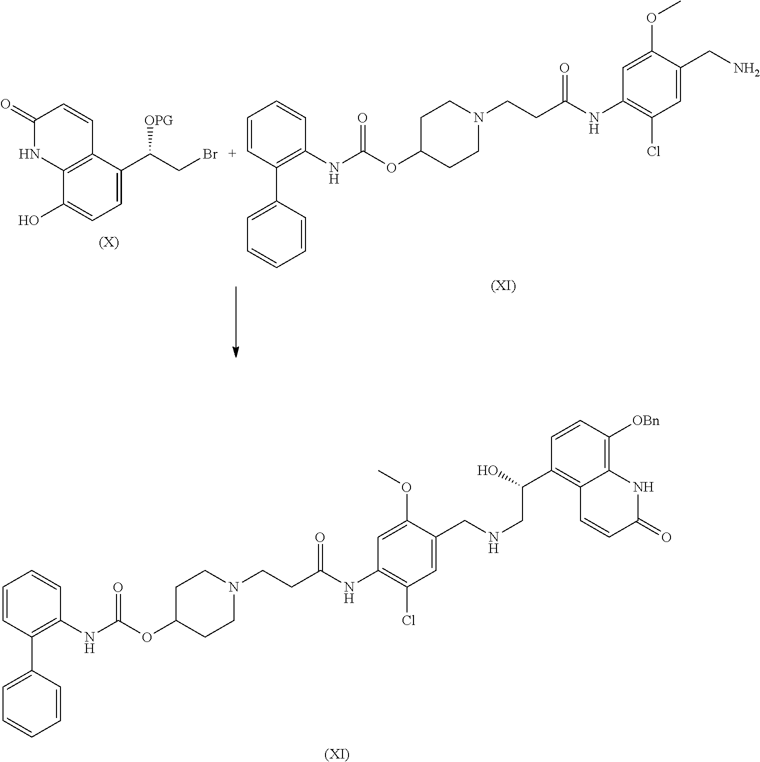

d) debenzylation of compound of formula XII in presence of debenzylating agent.

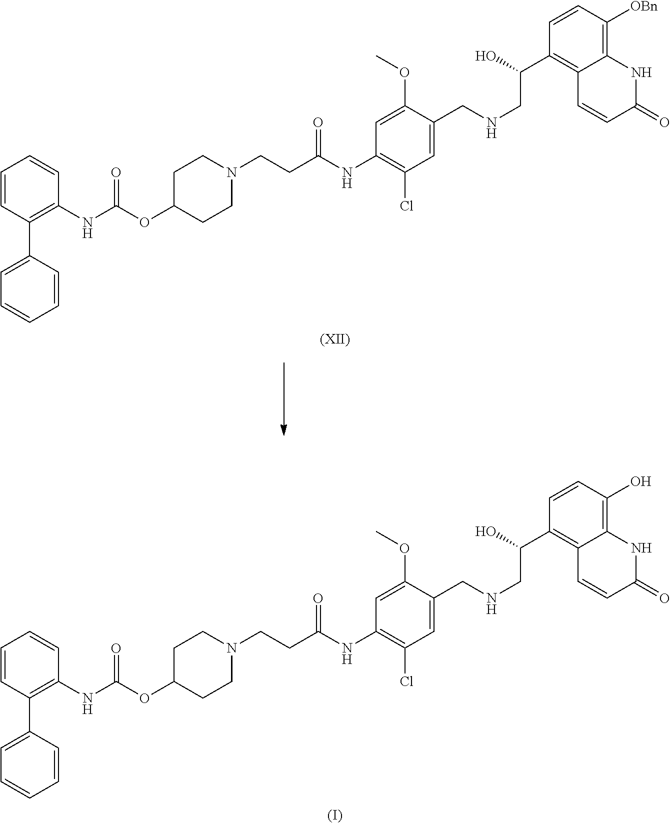

The detailed steps of the above-mentioned process as carried out under the solvent and reagent conditions are described herein:

In step a), cleavage of chiral epoxide is carried out in solvents selected from Tetrahydrofuran, or halo solvents, and the brominating agent is selected from bromine, 1,3-dibromohydantoin, tetra n-butyl ammonium tri bromide, NBS.

In step b), protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole.

In step c), condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

In step d), debenzylation is carried out using debenzylating agents selected from Pd/C, Pd/BaSO₄, or Raney nickel.

The Route 3 of synthesis of compound of the Formula (I) from the intermediate of compound of Formula (VII) via intermediates of the compound of Formula VII and compound of XI includes the steps of:
a) condensation of compound of formula VII with compound of formula XI in presence of base to form compound of formula XII;

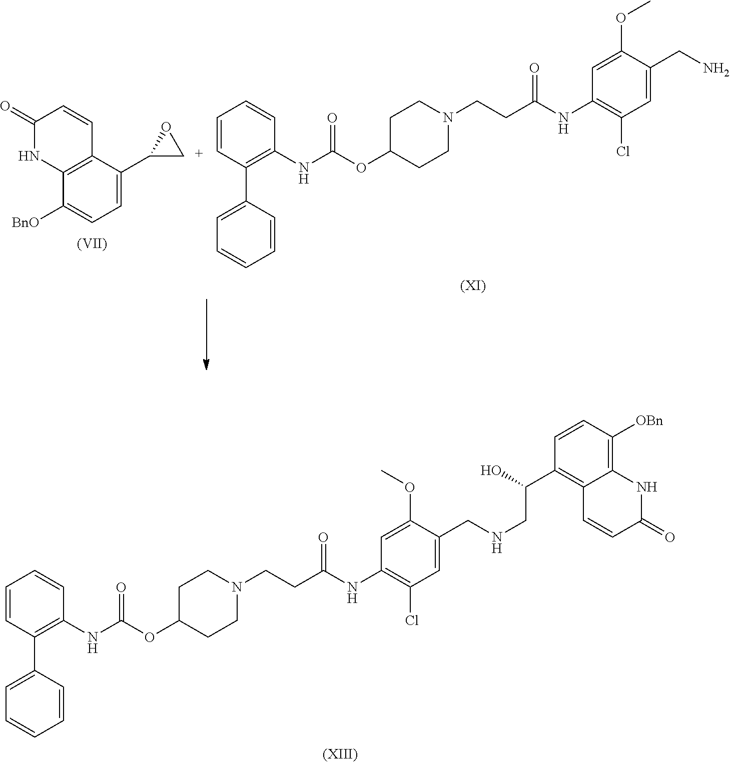

b) debenzylation of compound of formula XIII in presence of debenzylating agent.

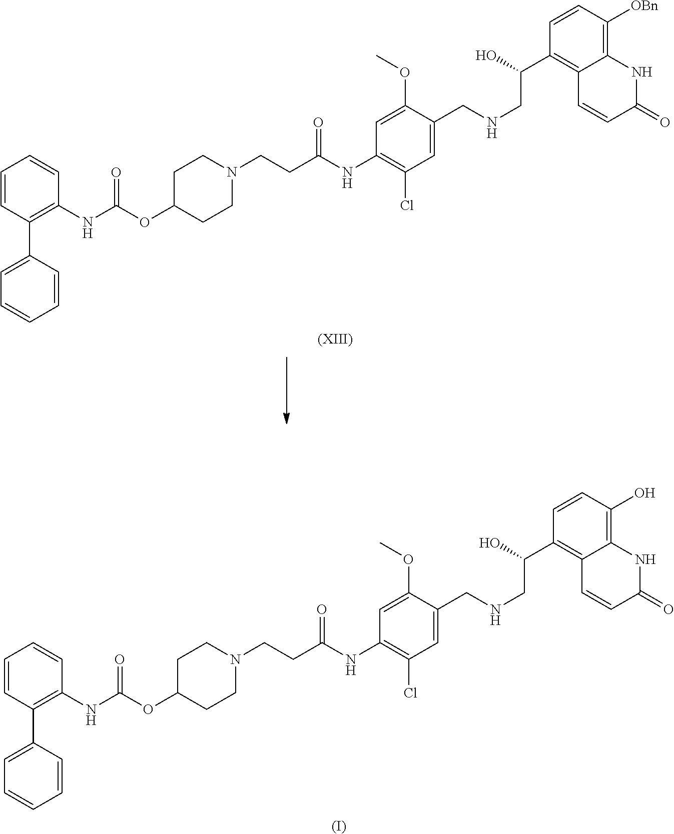

In step a), condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

In step b), debenzylation is carried out using debenzylating agents selected from Pd/C, Pd/BaSO₄, or Raney nickel.

The Route 4 of synthesis of compound of the Formula (I) from the intermediate of compound of Formula (VII) via intermediates of the compound of Formula XV and compound of Formula XVI includes the steps of: 5
a) cleavage of chiral epoxide compound of formula VII to form compound of formula XIV;

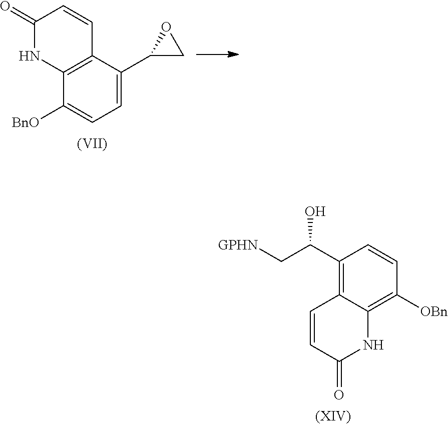

b) optionally protection of compound of formula XIV in presence of protecting agents to form compound of formula XV;

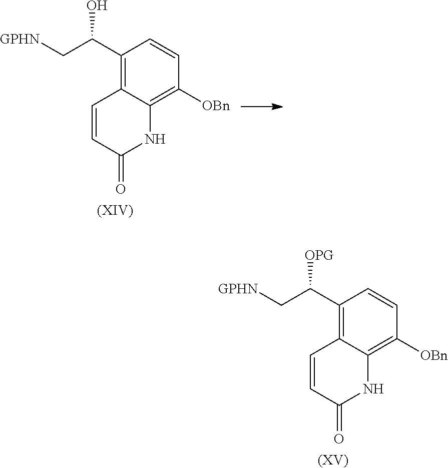

c) condensation of compound of formula XV with compound of formula XVI in presence of base to form compound of formula XVII;

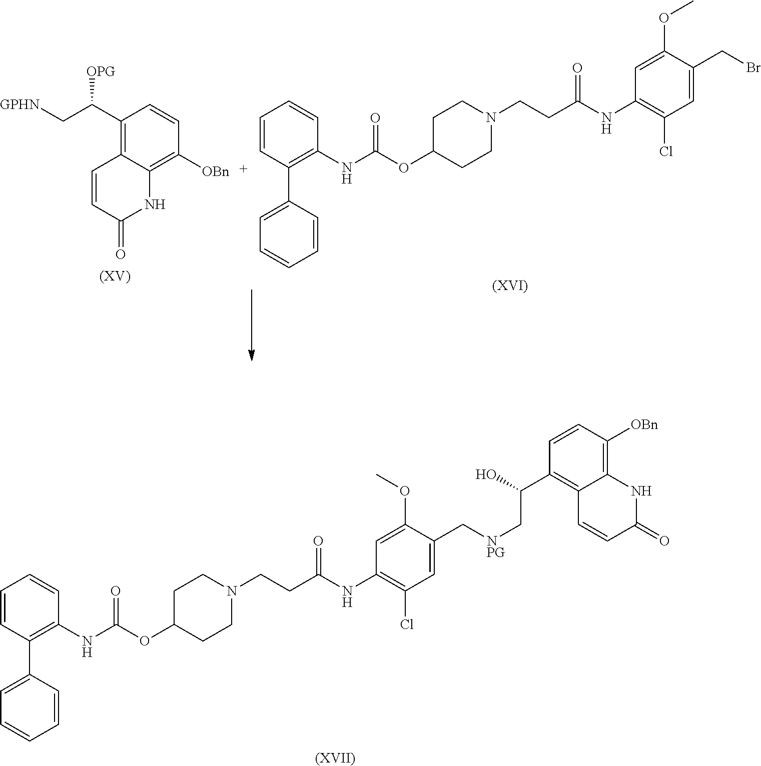

d) debenzylation of compound of formula XVII in presence of debenzylating agent.

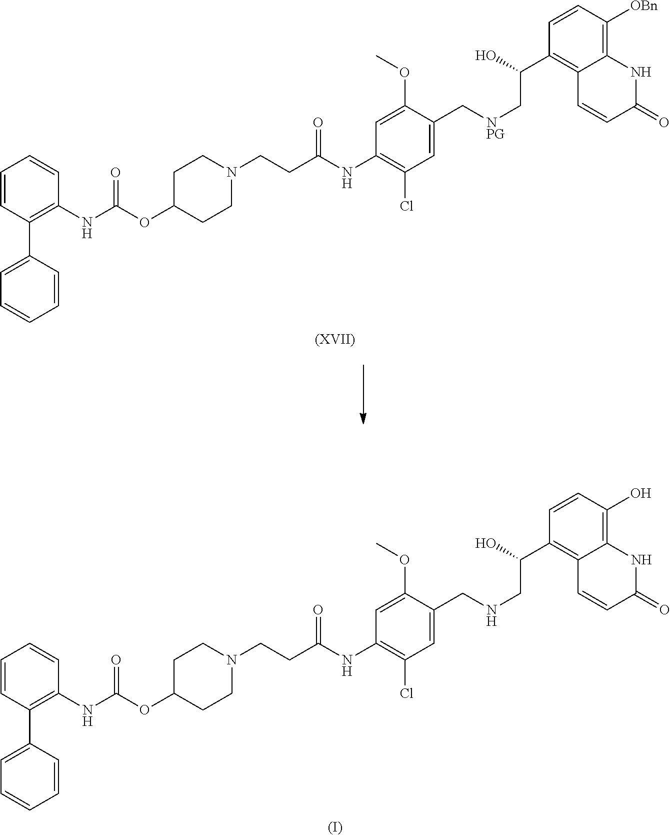

In step a), cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS.

In step b), protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole.

In step c), condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

In step d), debenzylation is carried out using debenzylating agents selected from Pd/C, Pd/BaSO₄, or Raney nickel.

The Route 5 of synthesis of compound of the Formula (I) from the intermediate of compound of Formula (VII) via intermediates of the compound of Formula XIX and compound of Formula XVI includes the steps of:
a) cleavage of chiral epoxide compound of formula VII to form compound of formula XVIII;

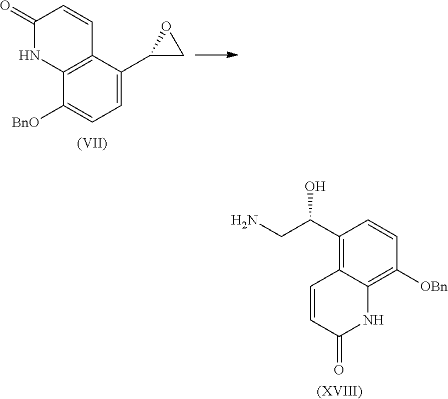

b) optionally protection of compound of formula XVIII in presence of protecting agents to form compound of formula XIX;

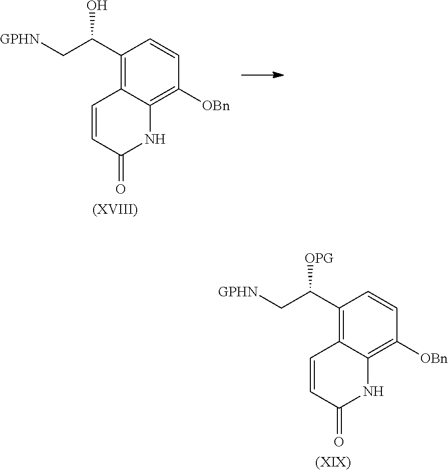

c) condensation of compound of formula XIX with compound of formula XVI in presence of base to form compound of formula XII;

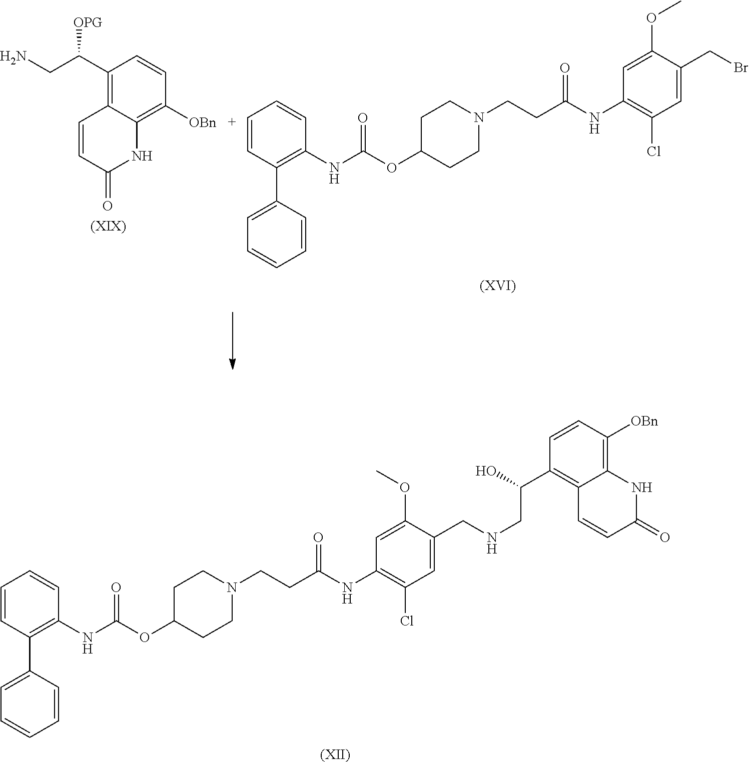

d) debenzylation of compound of formula XII in presence of debenzylating agent.

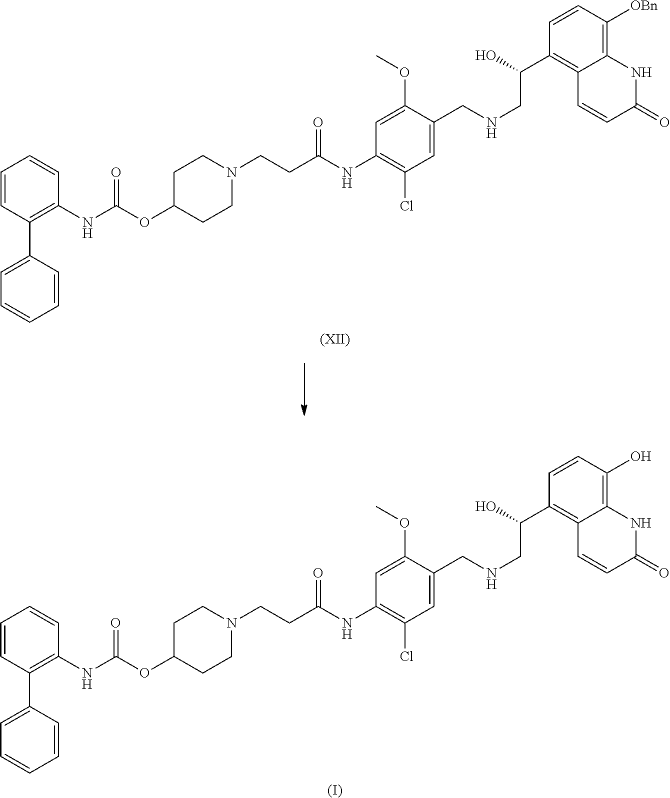

In step a), cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS.

In step b), protection is carried out by protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole.

In step c), condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

In step d), debenzylation is carried out using debenzylating agents selected from Pd/C, Pd/BaSO₄, or Raney nickel.

These and other embodiments will be apparent to those of skill in the art and others in view of the following detailed description of some embodiments. It should be understood, however, that this summary, and the detailed description illustrate only some examples of various embodiments, and are not intended to be limiting to the invention as claimed.

The process of the present invention is cost effective and results in high yield of the end product with maximum purity.

Advantageously, the process of the present invention does not use hypertoxic materials. The intermediate compounds prepared by the process are highly stable with high chemical purity. Further, the final compound (I) prepared by the process is highly efficient and possess excellent powdery nature.

### EXAMPLES

Only a few examples and implementations are disclosed. Variations, modifications, and enhancements to the described examples and implementations and other implementations can be made based on what is disclosed.

Examples are set forth herein below and are illustrative of different amounts and types of reactants and reaction conditions that can be utilized in practicing the disclosure. It will be apparent, however, that the disclosure can be practiced with other amounts and types of reactants and reaction conditions than those used in the examples, and the resulting devices various different properties and uses in accordance with the disclosure above and as pointed out hereinafter.

### Example 1: Preparation of Batefenterol by Route 1 Synthesis from its Intermediates of the Formula (X) and Formula (XD)

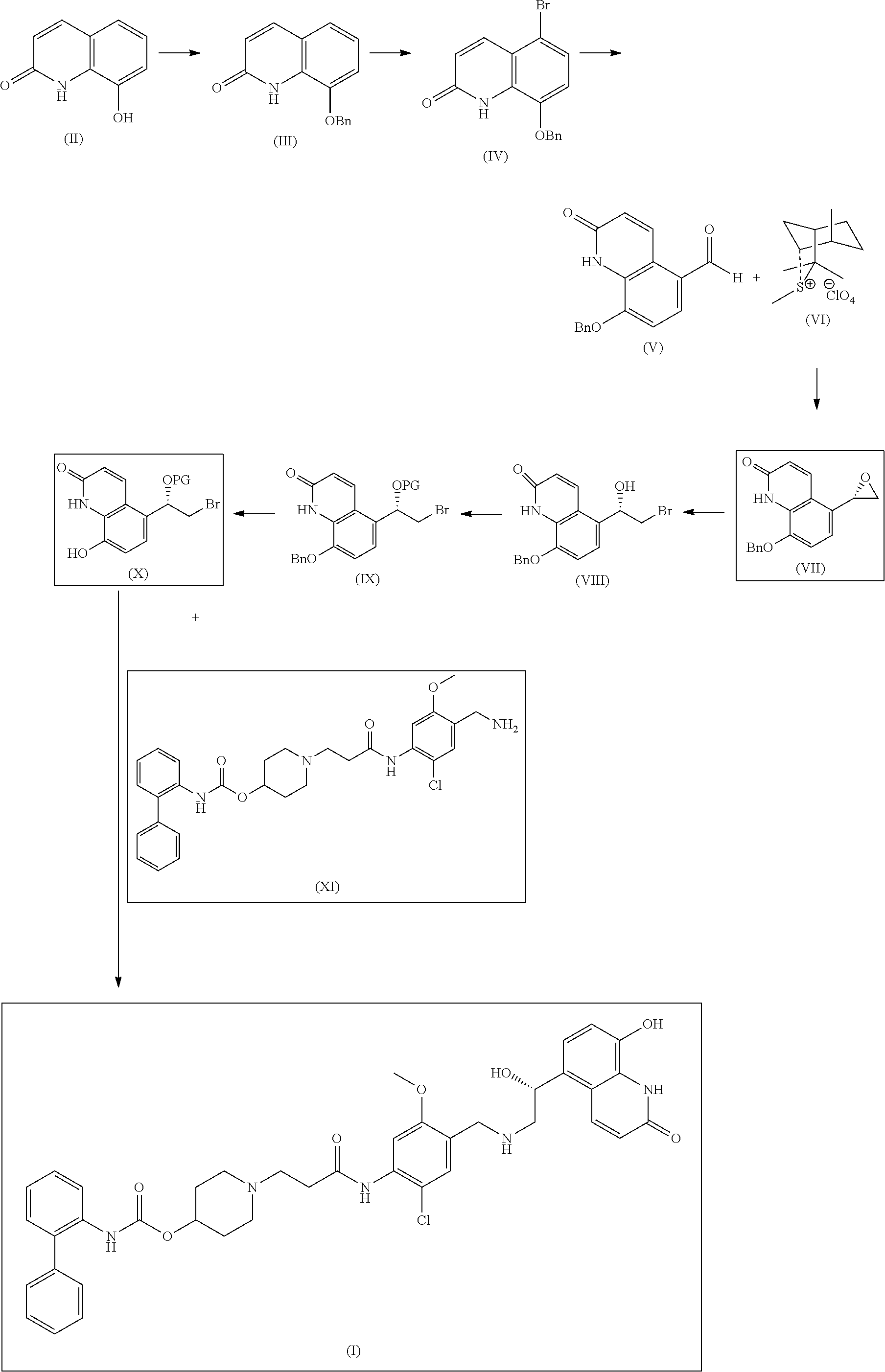 [0084] 1. To a solution of 8-Hydroxyquinolin-2-(1H)-one (II) (5.0 g, 0.031 mol) in acetone (40 mL) were added K₂CO₃ (5.1 g, 0.037 mol) and benzyl bromide (4.4 mL, 0.037 mol) at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S₂O₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.
5. A stirred solution of (VII) in THF was cooled to 10-15° C. and aq. HBr (48%1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound of Formula (VIII) was protected using suitable protecting agent to give protected product of (IX).
7. To the above compound (IX) (2.5 g) in 25 ml of methanol was added 10% Pd/C (250 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (X) (96% yield).
8. To a stirred solution of DMF (25 mL) and epoxide (X) (5.0 g) a solution of compound (XI) (10.07 g) in DMF 25 ml was added in a dropwise manner at room temperature under nitrogen. After complete addition, the mixture was allowed to warm to 50° C. and stirred for 4 h. The resulting homogeneous mixture was quenched with saturated aqueous Na₂SO₃ and extracted with ethyl acetate. The ethyl acetate layer was washed with water followed by saturated brine solution. The ethyl acetate layer was dried under anhydrous sodium sulphate at below 45° C. to get the crude compound (I) (72% yield). Recrystallisation of compound (I) gives 78% purity.

### Example 2: Preparation of Batefenterol by Route 2 Synthesis from its Intermediates of the Formula (IX) and Formula (XI)

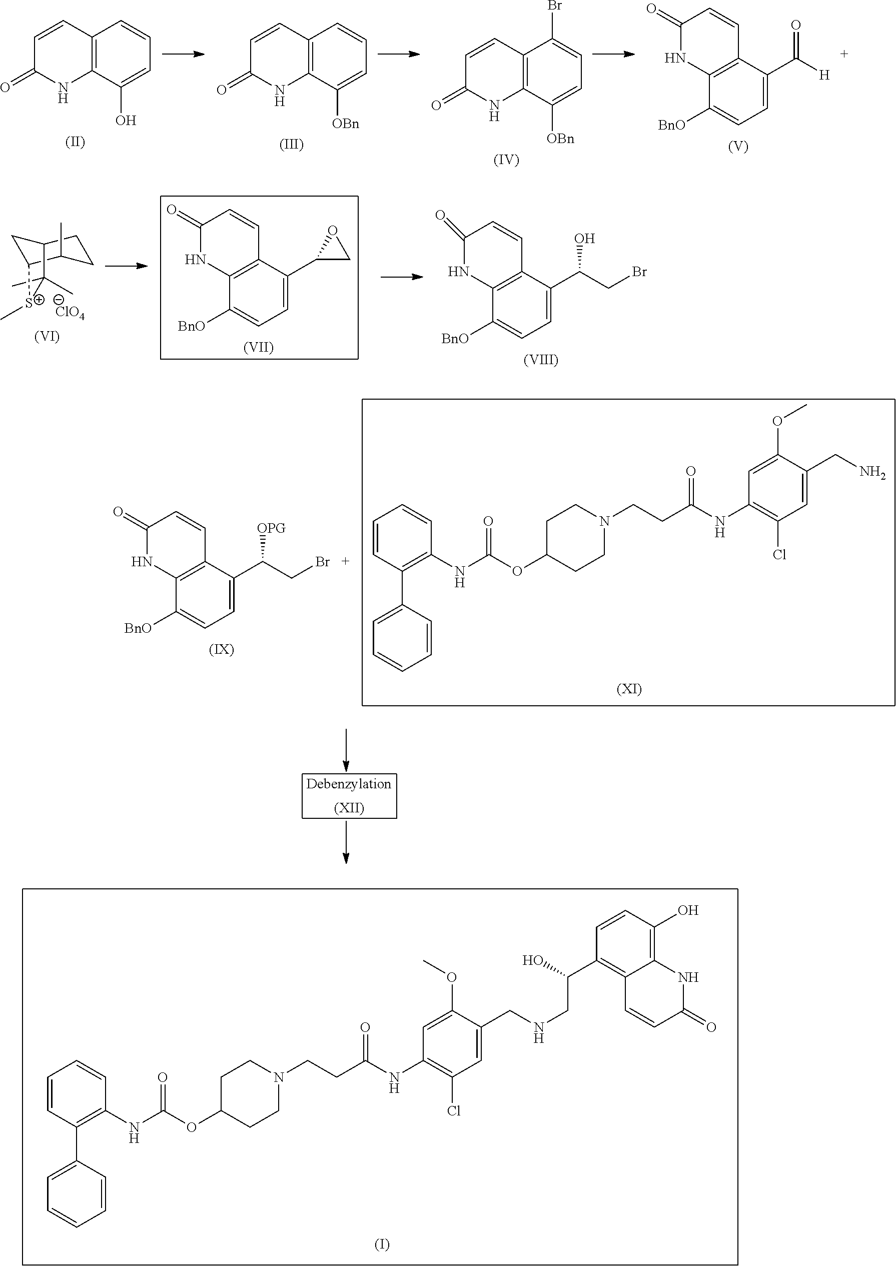 [0094] 1. To a solution of 8-Hydroxyquinolin-2-(1H)-one (II) (5.0 g, 0.031 mol) in acetone (40 mL) were added K₂CO₃ (5.1 g, 0.037 mol) and benzyl bromide (4.4 mL, 0.037 mol) at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S₂O₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.
5. A stirred solution of (VII) in THF was cooled to 10-15° C. and aq. HBr (48%1.1 m. eq) was added. The resulting mixture was stirred at the same temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound bromo hydrin (VIII).
6. The compound of Formula (VIII) is protected using suitable protecting agent to give protected product of (IX).
7. To a stirred solution of DMF (25 mL) and epoxide (IX) (5.0 g) a solution of compound (XI) (10.07 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with water followed by saturated brine solution. The ethyl acetate layer was dried under anhydrous sodium sulphate at below 45° C. to get the crude compound (XII) (72% yield).
8. To the above compound (XII) (3.5 g) in 35 ml of methanol was added 10% Pd/C (350 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (I) (86% yield). Recrystallisation of compound (I) gives 82% purity.

### Example 3: Preparation of Batefenterol by Route 3 Synthesis from its Intermediates of the Formula (VII) and Formula (XI)

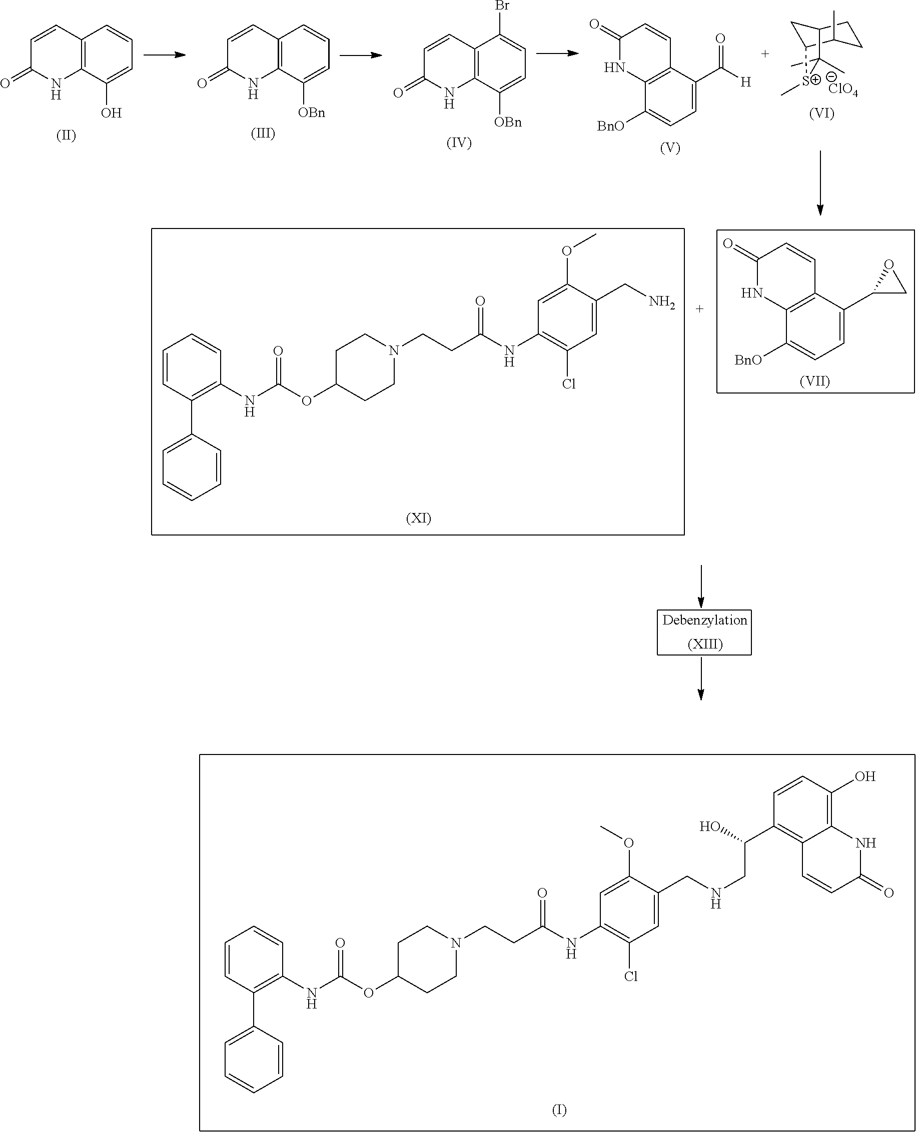 [0103] 1. To a solution of 8-Hydroxyquinolin-2-(1H)-one (II) (5.0 g, 0.031 mol) in acetone (40 mL) were added K₂CO₃ (5.1 g, 0.037 mol) and benzyl bromide (4.4 mL, 0.037 mol) at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S₂O₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.
5. To a stirred solution of DMF (25 mL) and epoxide (VII) (5.0 g) a solution of compound (XI) (9.5 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with water followed by saturated brine solution. The ethyl acetate layer was dried under anhydrous sodium sulphate at below 45° C. to get the crude compound (XIII) (78% yield).
6. To the above compound (XII) (2.5 g) in 25 ml of methanol was added 10% Pd/C (250 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (I) (86% yield). Recrystallisation of compound (I) gives 82% purity.

### Example 4: Preparation of Batefenterol by Route 4 Synthesis from its Intermediates of the Formula (XV) and Formula (XVI)

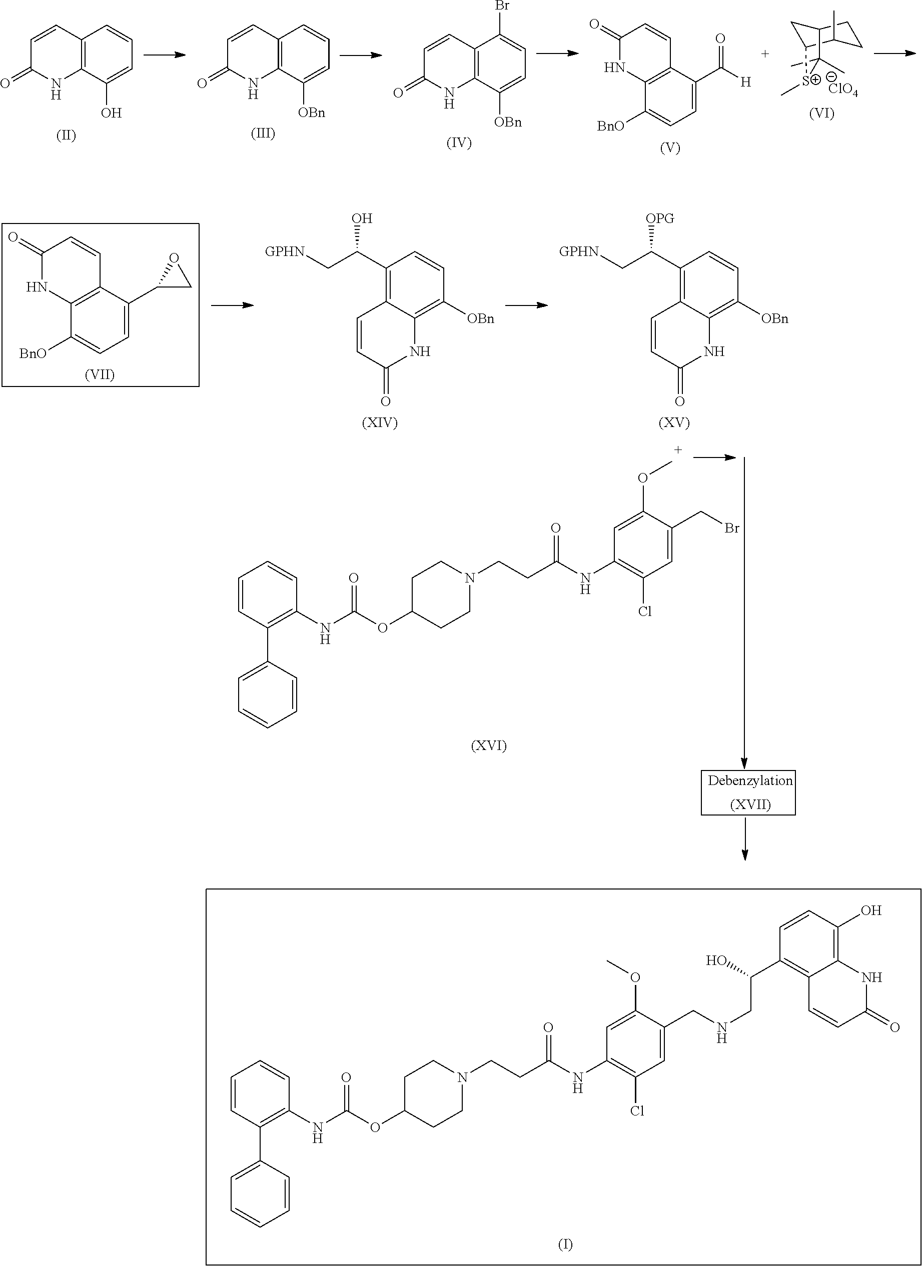 [0111] 1. To a solution of 8-Hydroxyquinolin-2-(1H)-one (II) (5.0 g, 0.031 mol) in acetone (40 mL) were added K₂CO₃ (5.1 g, 0.037 mol) and benzyl bromide (4.4 mL, 0.037 mol) at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S₂O₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.
5. A stirred solution of (VII) in THF was cooled to 10-15° C. and amine derivative 1.1 m. eq was added. The resulting mixture was stirred at the room temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound (XIV).
6. The compond (XIV) is protected using suitable protecting agent to give protected product of (XV).
7. To a stirred solution of DMF (25 mL) and epoxide (XV) (5.0 g) a solution of compound (XVI) (9.5 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with water followed by saturated brine solution. The ethyl acetate layer was dried under anhydrous sodium sulphate at below 45° C. to get the crude compound (XVII) (75% yield).
8. To the above compound (XVII) (3.5 g) in 35 ml of methanol was added 10% Pd/C (350 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (I) (82% yield). Recrystallisation of compound (I) gives 82% pure.

### Example 5: Preparation of Batefenterol by Route 5 Synthesis from its Intermediates of the Formula (XIX) and Formula (XVI)

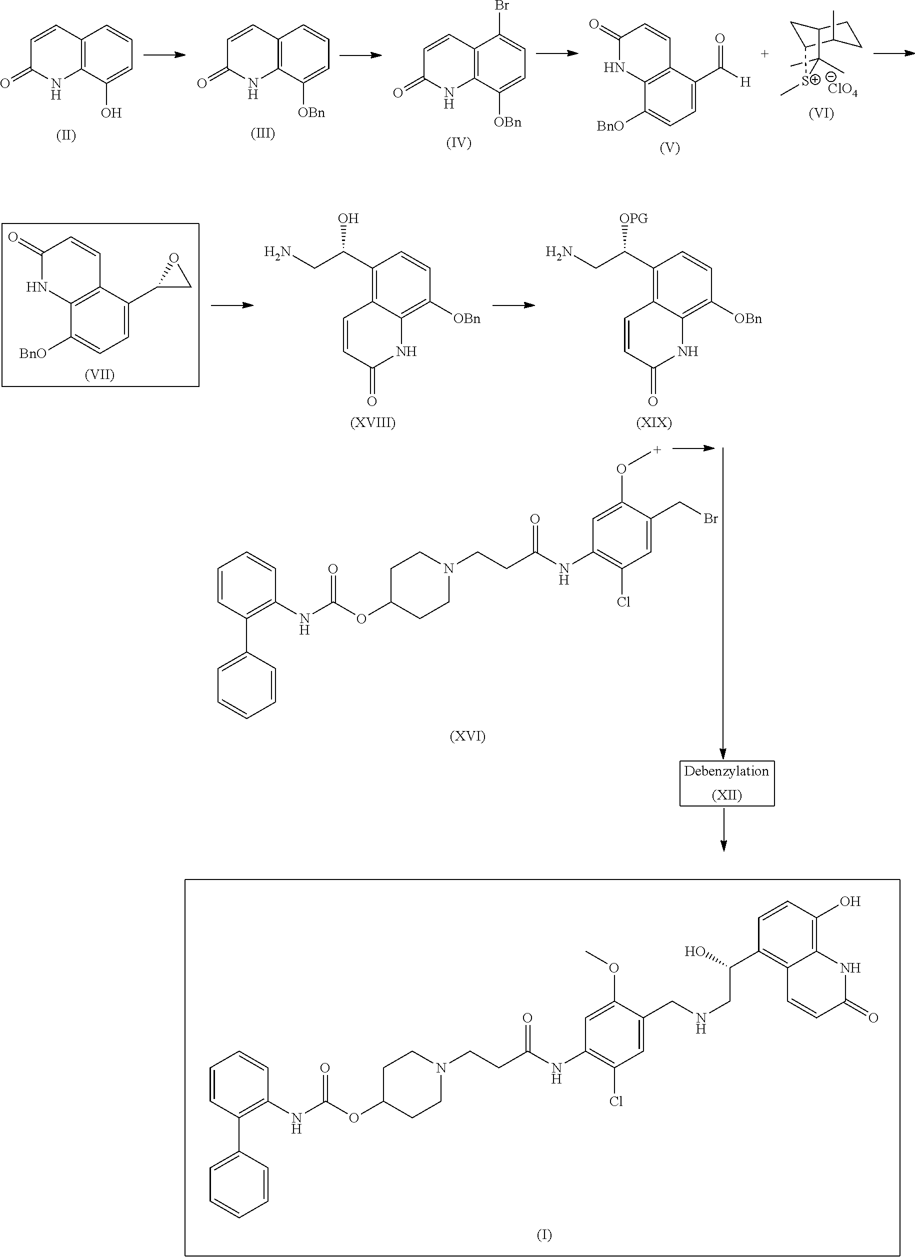 [0121] 1. To a solution of 8-Hydroxyquinolin-2-(1H)-one (II) (5.0 g, 0.031 mol) in acetone (40 mL) were added K₂CO₃ (5.1 g, 0.037 mol) and benzyl bromide (4.4 mL, 0.037 mol) at 0° C. The mixture was stirred at reflux temperature under nitrogen. After completion, the solvent was removed under reduced pressure and quenched with 1N HCl and extracted with ethyl acetate (3×50 mL), dried over sodium sulphate, filtered
and distilled under reduced pressure. The residue was purified by silica gel chromatography to give the benzyl ether (III) as a colourless solid in 89% yield.
2. A solution of bromine (0.5 mL, 0.02 mol) in acetic acid (6.0 mL) was added dropwise to a solution of (III) (4.5 g, 0.018 mol) in acetic acid (40 mL). The mixture was stirred at room temperature for 4 h and quenched with sat. Na₂S₂O₃ solution and then extracted with EtOAc (3×40 mL). The combined organic layers were washed with sat. NaHCO₃ solution followed by water and brine solution and dried over Na₂SO₄. The solvent was evaporated and the resulting residue was purified by flash chromatography to afford the compound (IV) (80% yield) as a colourless solid.
3. The above bromo acetonide (IV) (3.7 g, 0.011 mol.) was dissolved in dry THF, cooled to −78° C. and a 1.6 M solution of n-butyl lithium (14.1 mL, 1.6 M in hexane, 0.022 mol.) was added drop wise. The mixture was stirred at −78° C. for 2 h and then treated with dry N, N-dimethylformamide (12.7 mL, 0.17 mol) as a 1:1 solution in dry THF. The resulting mixture was stirred at −78° C. for 0.75 h and warmed slowly up to room temperature. Then the mixture was diluted with diethyl ether, washed with water followed by a brine solution and dried over MgSO₄. The solvent was removed in vacuo and the residue was purified by column chromatograph on silica gel to give the required compound (V) (74% yield).
4. A mixture of aldehyde (V) (2.5 g, 0.0089 mol), sulfonium perchlorate (VI) (3.2 g, 0.011 mol) and powdered KOH (0.62 g, 0.011 mol) in tert-butyl alcohol (40 mL) was stirred at room temperature for 48 h. Then the mixture was quenched with water and extracted with dichloromethane (30 mL×3). The combined extracts were washed with sat. NaCl and dried over MgSO₄. Evaporation of the solvent followed by purification on silica gel using a mixture of EtOAc-hexane as an eluent gave the epoxide (VII) in 80% yield.
5. A stirred solution of (VII) in THF was cooled to 10-15° C. and amine derivative 1.2 m. eq was added. The resulting mixture was stirred at the room temperature till reaction completion. Then the reaction mixture was added to chilled water and extracted into ethyl acetate. The ethyl acetate layer was washed with water followed by saturated brine solution. The ethyl acetate layer was dried under anhydrous sodium sulphate. The ethyl acetate is distilled under vacuum at below 45° C. to get the compound (XVIII).
6. The compound of formula (XVIII) is protected using suitable protecting agent to give protected product of (XIX).
7. To a stirred solution of DMF (25 mL) and compound (XIX) (5.0 g) a solution of compound (XVI) (9.5 g) in DMF 25 ml was added in a dropwise manner at room temperature under nitrogen. After complete addition, the mixture was allowed to warm to 50-60° C. and stirred for 4-6 h. The resulting homogeneous mixture was quenched with saturated aqueous Na₂SO₃ and extracted with ethyl acetate. The ethyl acetate layer was washed with water followed by saturated brine solution. The ethyl acetate layer was dried under anhydrous sodium sulphate at below 45° C. to get the crude compound (XII) (78% yield).
8. To the above compound (XII) (3.5 g) in 35 ml of methanol was added 10% Pd/C (350 mg). The solution was placed in a stainless-steel reactor, which was then charged with hydrogen gas (150 psi). After being stirred for 24 h at room temperature, the mixture was filtered through Celite to remove the catalyst. The filtrate thus obtained was evaporated to give the product (I) (86% yield). Recrystallisation of compound (I) gives 82% pure.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. A process for preparation of Batefenterol having the Formula (I) 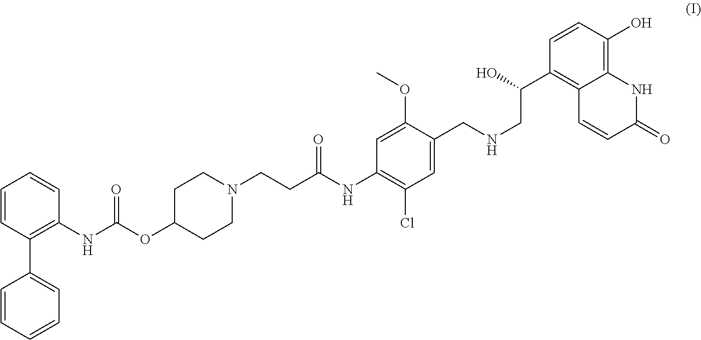 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]
or pharmaceutically acceptable salts thereof, and its chiral intermediates, comprising the steps of:
a) addition of the compound of Formula (II) to a solvent followed by adding a base and a benzylating agent to obtain a compound of Formula (II); 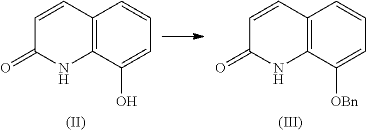 [SMILES: O=c1ccc2cccc(O)c2[nH]1 ; O=c1ccc2cccc(OCc3ccccc3)c2[nH]1 ; "(II)" ; "(III)"]
b) addition of the compound of Formula (III) to an acid and a brominating agent to make a reaction mixture; to obtain a compound of Formula (IV); 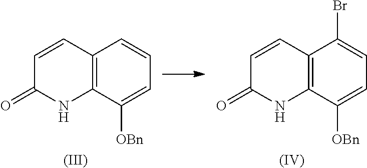 [SMILES: O=c1ccc2cccc(OCc3ccccc3)c2[nH]1 ; O=c1ccc2c(Br)ccc(OCc3ccccc3)c2[nH]1 ; "(III)" ; "(IV)"]
c) carbonylation of compound of formula IV with strong base to give compound of formula V; 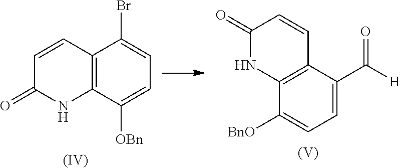
d) chiral epoxidation of compound of formula V, using chiral sulphide compound of Formula (VI) to form compound of formula VII; 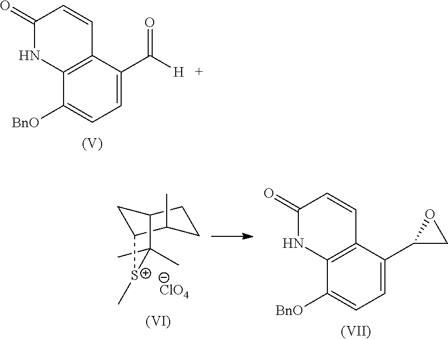
e) synthesis of Batefenterol having the Formula (I) from the compound of Formula (VII):
(i) via intermediate compounds of Formula (X) and Formula (XI); or
(ii) via intermediate compounds of the Formula (IX) and Formula (XI); or
(iii) via intermediate compounds of the Formula (VII) and Formula (XI); or
(iv) via intermediate compounds of the Formula (XV) and Formula (XVI); or
(v) via intermediate compounds of the Formula (XIX) and Formula (XVI).

2. The process as claimed in claim 1, wherein:
a) carbonylation is carried out in presence of the solvents selected from tetrahydrofuran, methyl tert-butyl ether, diisopropyl ether, or diethyl ether; followed by treating with a strong base selected from n-butyl lithium, s-butyl lithium, lithium diisopropylamide, potassium bis(trimethylsilyl)amide; further followed by addition of a mixture of solvents, N, N-dimethylformamide and tetrahydrofuran in the ratio of 1:1 at the temperature of about −78° C. to 0° C.;
b) chiral epoxidation is carried out using chiral sulphide derivative (VI), in presence of base selected from potassium hydroxide, sodium hydroxide, lithium hydroxide, in solvents selected from tert-butyl alcohol, isopropyl alcohol, methanol.

3. A process as claimed in claim 1, wherein synthesis of Batefenterol from intermediate compound of Formula (VII) via intermediate compounds of Formula (X) and Formula (XI) includes the following steps:
a) cleavage of chiral epoxide compound of formula VII is brominating agent to form compound of formula VIII; 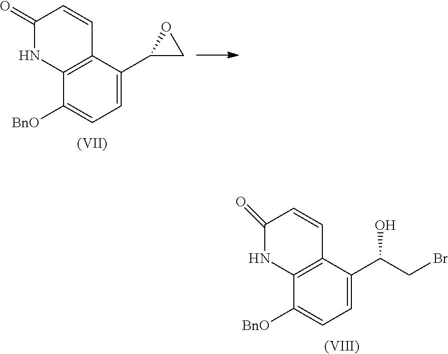
b) optionally protection of compound of formula VIII in presence of protecting agents to form compound of formula IX; 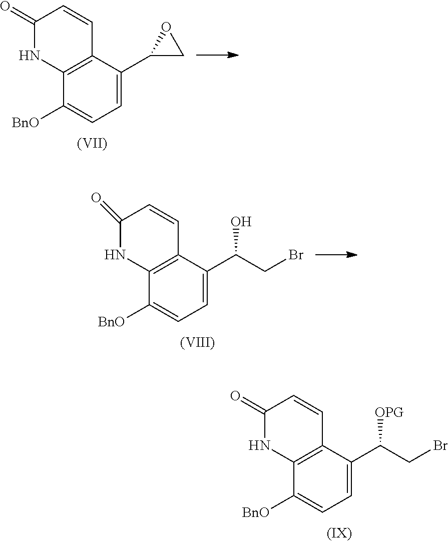
c) debenzylation of compound of formula IX in presence of debenzylating agent to form compound of formula X; 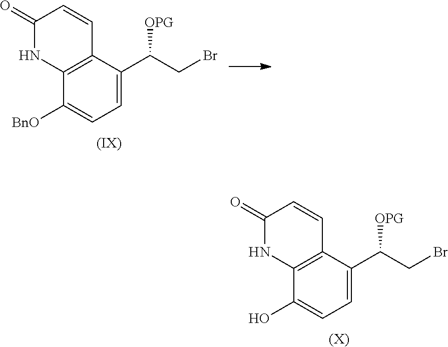
d) condensation of compound of formula X with compound of formula XI in presence of base.

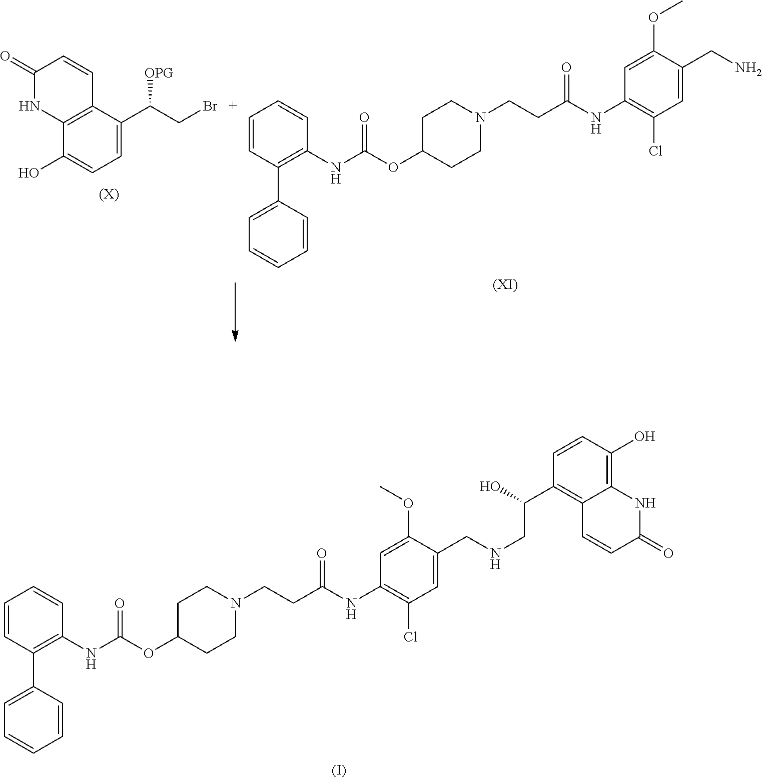

4. The process as claimed in claim 3, wherein the cleavage of chiral epoxide is carried out in solvents selected from Tetrahydrofuran, or halo solvents, and brominating agents selected from bromine, 1,3-dibromohydantoin, tetra n-butyl ammonium tri bromide, NBS; the protection is carried out using protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; the debenzylation is carried out using debenzylating agents selected from Pd/C, Pd/BaSO₄, or Raney nickel; the condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃.

5. A process as claimed in claim 1, wherein synthesis of Batefenterol from intermediate compound of Formula (VII) via intermediate compounds of the Formula (IX) and Formula (XI) includes the following steps:
a) cleavage of chiral epoxide compound of formula VII is brominating agent to form compound of formula VIII; 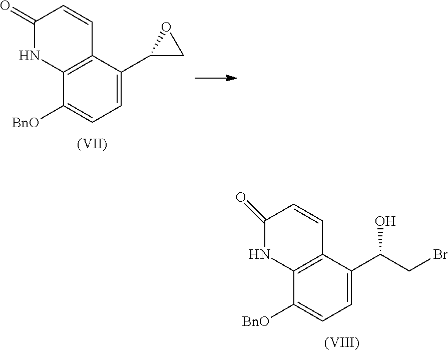
b) optionally protection of compound of formula VIII in presence of protecting agents to form compound of formula IX; 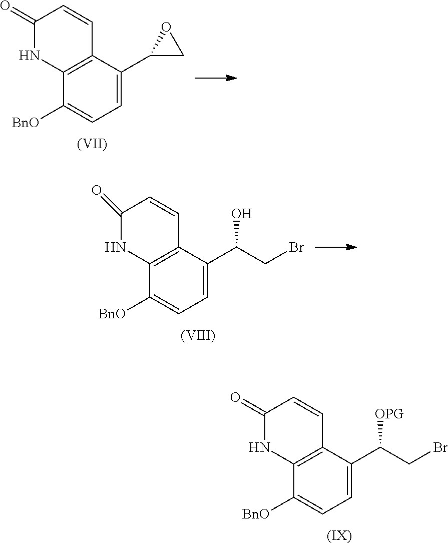
c) condensation of compound of formula IX with compound of formula XI in presence of base to form compound of formula XII; 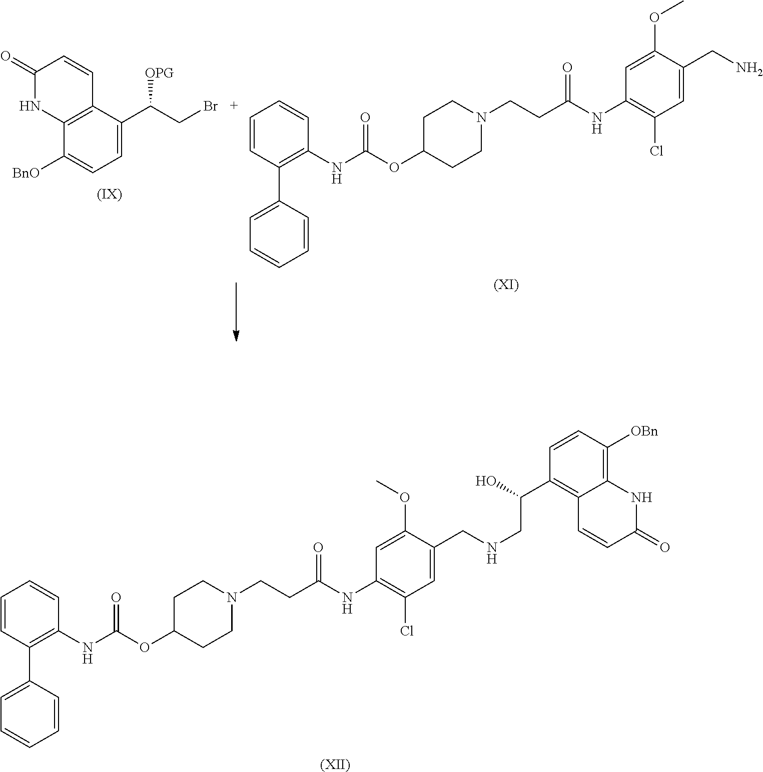
d) debenzylation of compound of formula XII in presence of debenzylating agent.

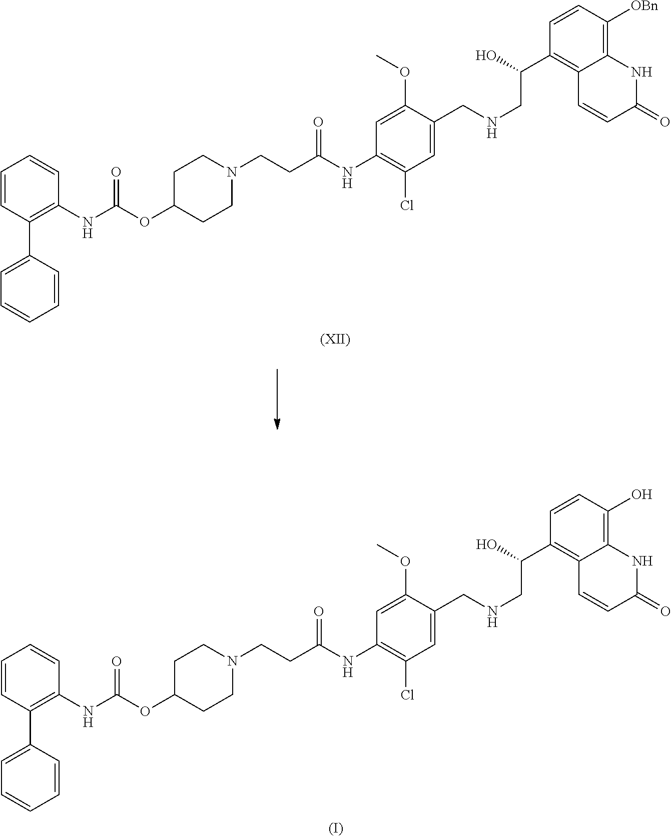

6. The process as claimed in claim 5, wherein the cleavage of chiral epoxide is carried out in solvents selected from Tetrahydrofuran, or halo solvents, and the brominating agent is selected from bromine, 1,3-dibromohydantoin, tetra n-butyl ammonium tri bromide, NBS; the protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; the condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; the debenzylation is carried out using debenzylating agents selected from Pd/C, Pd/BaSO₄, or Raney nickel.

7. A process as claimed in claim 1, wherein synthesis of Batefenterol from intermediate compound of Formula (VII) via intermediate compounds of the Formula (VII) and Formula (XI) includes the following steps:
a) condensation of compound of formula VII with compound of formula XI in presence of base to form compound of formula XII; 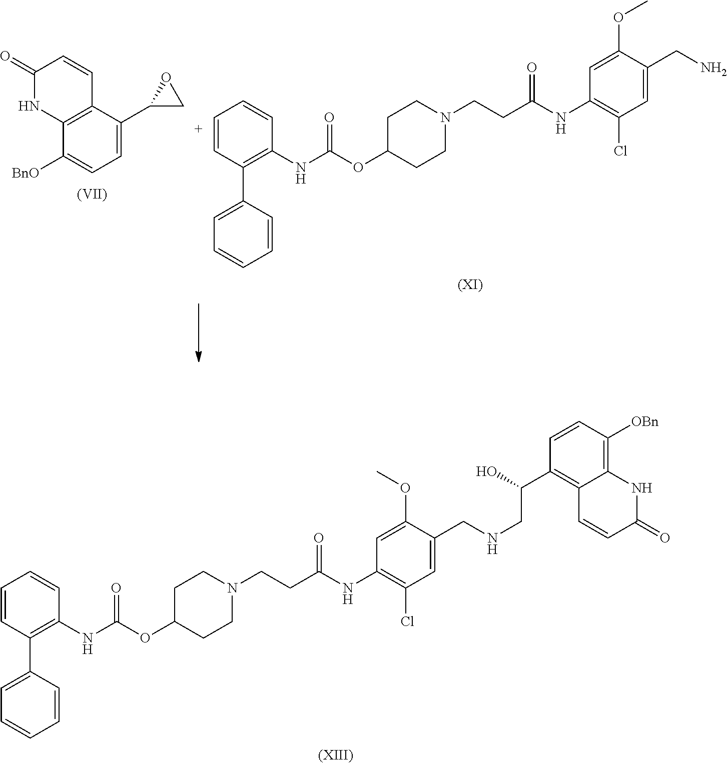
b) debenzylation of compound of formula XIII in presence of debenzylating agent.

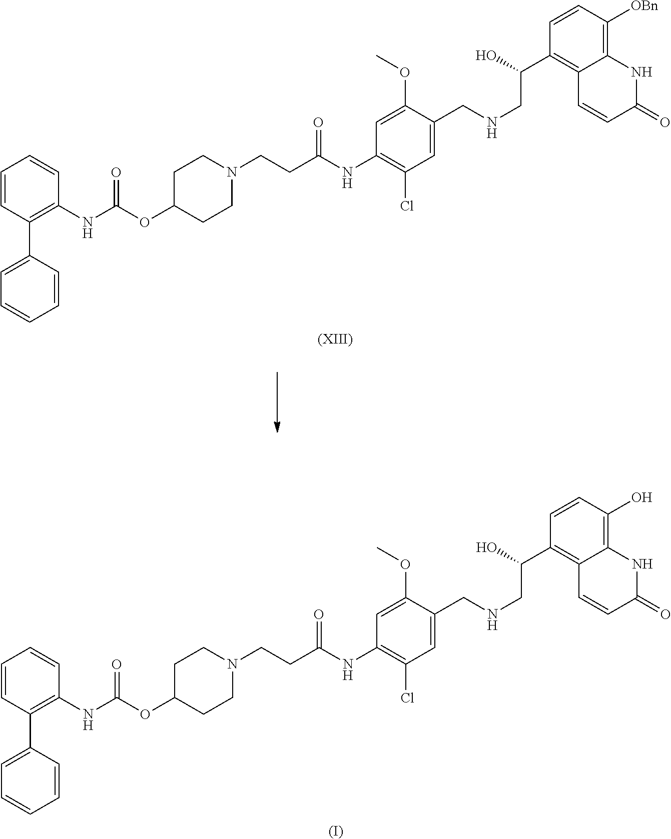

8. The process as claimed in claim 7, wherein condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; and debenzylation is carried out using debenzylating agents selected from Pd/C, Pd/BaSO₄, or Raney nickel.

9. A process as claimed in claim 1, wherein synthesis of Batefenterol from intermediate compound of Formula (VII) via intermediate compounds of the Formula (XV) and Formula (XVI) includes the following steps:
a) cleavage of chiral epoxide compound of formula VII to form compound of formula XIV; 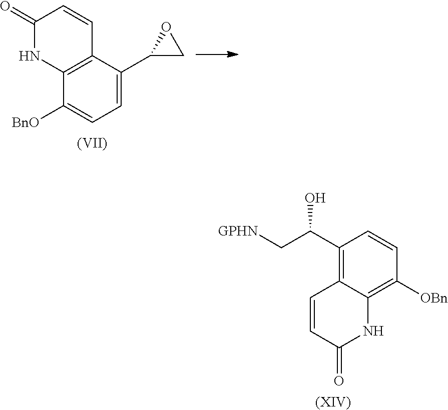
b) optionally protection of compound of formula XIV in presence of protecting agents to form compound of formula XV; 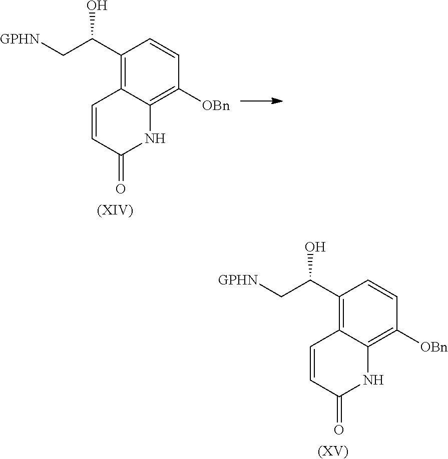
c) condensation of compound of formula XV with compound of formula XVI in presence of base to form compound of formula XVII; 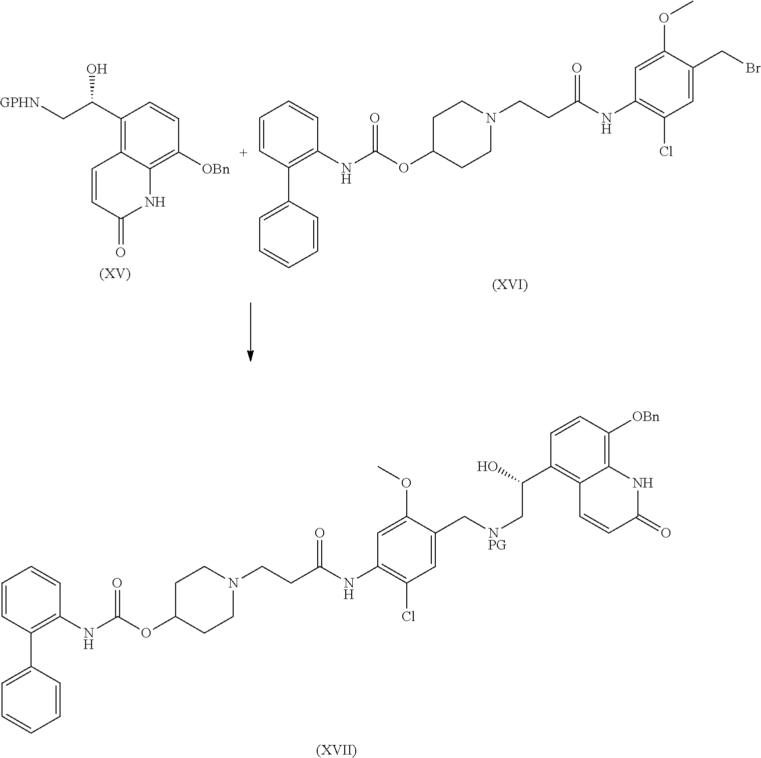
d) debenzylation of compound of formula XVII in presence of debenzylating agent.

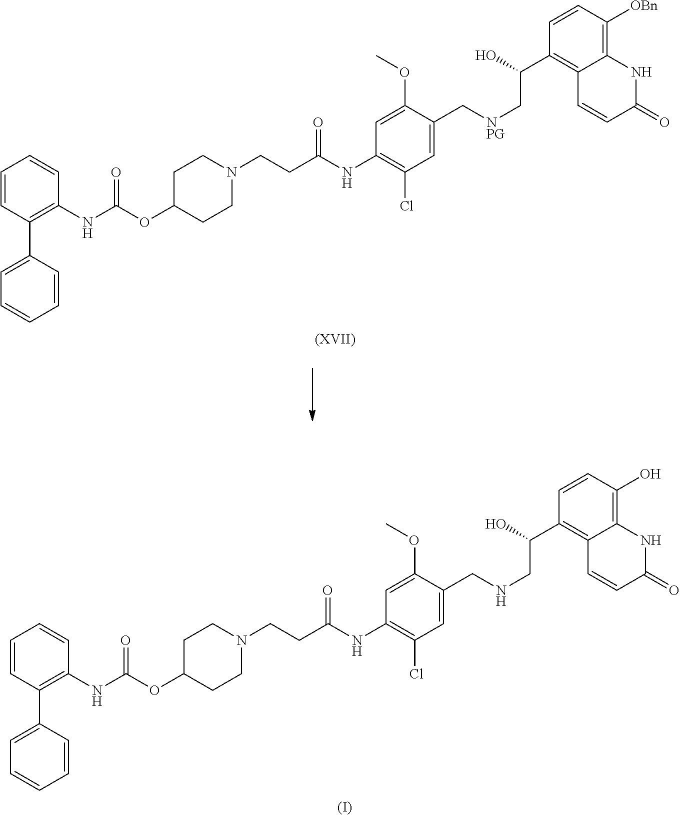

10. The process as claimed in claim 9, wherein cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS; protection is carried out in presence of protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃, debenzylation is carried out using debenzylating agents selected from Pd/C, Pd/BaSO₄, or Raney nickel.

11. A process as claimed in claim 1, wherein synthesis of Batefenterol from intermediate compound of Formula (VII) via intermediate compounds of the Formula (XIX) and Formula (XVI) includes the following steps:
a) cleavage of chiral epoxide compound of formula VII to form compound of formula XVIII; 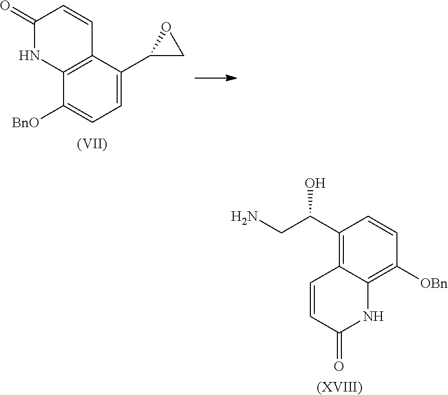
b) optionally protection of compound of formula XVIII in presence of protecting agents to form compound of formula XIX; 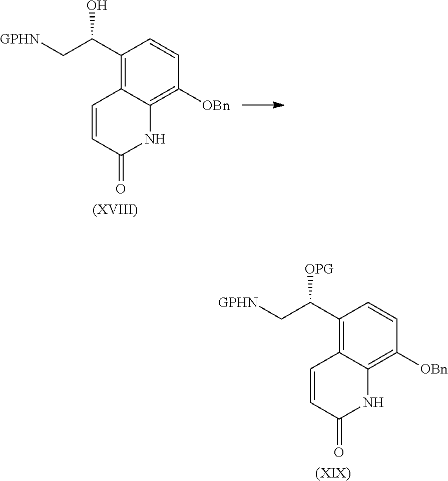
c) condensation of compound of formula XIX with compound of formula XVI in presence of base to form compound of formula XI; 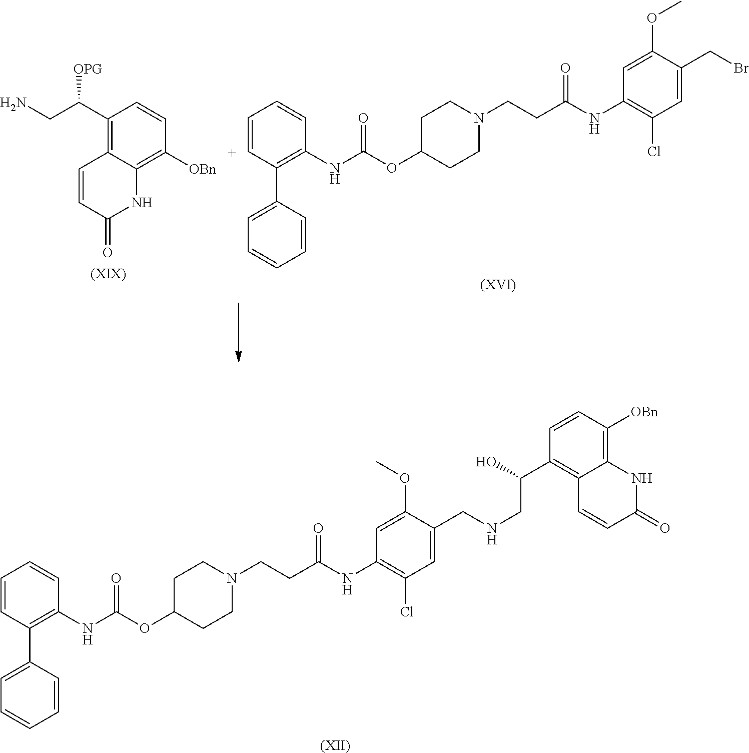
d) debenzylation of compound of formula XII in presence of debenzylating agent.

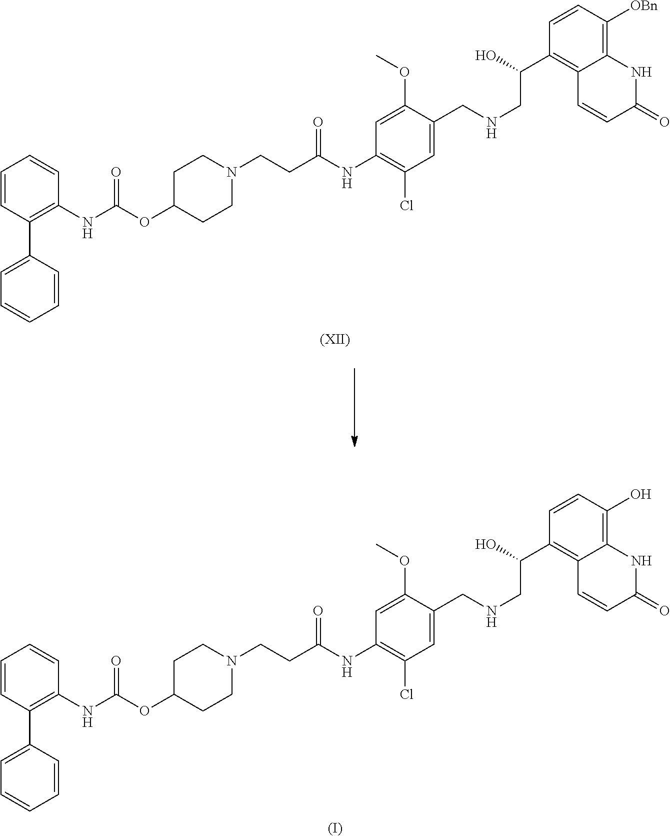

12. The process as claimed in claim 11, wherein cleavage of chiral epoxide is carried out in solvents selected from Tetrahydrofuran, or halo solvents, and the brominating agent selected from bromine, 1,3-dibromohydantoin, tetra n-butyl ammonium tri bromide, NBS; protection is carried out by protecting agents selected from THP, TBDMS, TMS and benzyl bromide, benzyl chloride, benzyl iodide, in solvents selected from acetone, THF, DMF, acetonitrile, 2-methyl THF, MIBK, halo solvents using base selected from K₂CO₃, Na₂CO₃, Cs₂CO₃, imidazole; condensation is carried out in presence of solvents selected from acetone, THF or DMF, acetonitrile, 2-methyl THF, MIBK and base selected from group of K₂CO₃, Na₂CO₃, NaOH, KOH or Cs₂CO₃; debenzylation is carried out using debenzylating agents selected from Pd/C, Pd/BaSO₄, or Raney nickel.
